(19) United States
(12) Patent Application Publication
(10) Pub. No.: US 2012/0251511 A1
(43) Pub. Date: Oct. 4, 2012

(54) COMPOSITION AND PRODUCTS FOR ENABLING THE PRODUCTION OF EQUOL IN VIVO

(76) Inventors: Eric Donald Murphy, Charlottesville, VA (US); Lowell Ernest Owen, Omaha, NE (US)

(21) Appl. No.: 13/433,080

(22) Filed: Mar. 28, 2012

Related U.S. Application Data
(60) Provisional application No. 61/468,541, filed on Mar. 28, 2011.

(51) Int. Cl. A61K 35/74; A61P 5/24; A61P 19/00; A61P 15/12

(52) U.S. Cl. 424/93.45; 424/93.4; 424/93.46

(57) ABSTRACT

The isoflavone equol has the ability to mimic estrogen at the receptor level, as well as the ability to mitigate the deleterious effects of dihydrotestosterone (DHT) in vivo, thus resulting in potential health benefits for both men and women. Only an approximated one-third of the global population has the inherent ability to produce equol. Diet and colonic bacterial composition play a large role in natural equol production. The present invention can increase the probability of producing significant quantities of equol in vivo. Experimentation has shown the repeatable and measurable evidence of equol. Data from male test subjects indicates clinically significant positive effects on androgenic alopecia and acne vulgaris.

### CROSS-REFERENCE TO RELATED APPLICATIONS

This application claims priority to U.S. Provisional Application No. 61/468,541 filed Mar. 28, 2011, the contents of which are incorporated herein by reference in its entirety.

### BACKGROUND OF THE INVENTION

1. Field of the Invention

The present invention relates to compositions using equol produced from daidzin, via daidzein, or daidzein itself in the colon (intestines, digestive tract, gut) in the treatment of certain health conditions.

2. Description of Related Art

Equol, specifically the enantiomer S-equol (“equol”), has attracted much interest in the research community as a potent isoflavone, with a high affinity for the estrogen receptor. This has made equol a prime focus for the treatment of various conditions, including menopausal symptoms. Equol also has a secondary attribute that makes it more functionally relevant to both males and females; it binds dihydrotestosterone (DHT) in vivo. Hence, equol has the ability to positively impact the health of both men and women. S-equol is a naturally occurring isoflavone that is biotransformed from daidzein by bacterial flora in the colon. The colonic flora only produces the natural S-equol enantiomer.

The vast majority of the world's population is unable to produce equol endogenously for two reasons. Most individuals do not regularly ingest foods or supplements that contain isoflavones, specifically daidzin (the glucoside of daidzein). Also, most individuals do not have the correct balance of colonic bacteria that is needed to produce equol from daidzein. These two reasons apply particularly to people who consume a Western-style diet high in saturated fats. Typically, a Southeastern Asian diet is much more conducive to endogenous equol production. It seems as though Southeastern Asian diets help attract and proliferate bacteria in the colon that are critical to equol production. Roughly half of the population of Korea and Japan are equol producers, versus one-third of the global population.

It is important to understand that simply consuming a traditional Southeastern Asian diet may not induce equol production. Environmental, health and possibly genetic factors are also relevant to equol producing capability. The purpose of the present invention is to afford both men and women of any race or ethnicity the opportunity to produce beneficial levels of equol endogenously.

### BRIEF SUMMARY OF THE INVENTION

Briefly described, in exemplary form, the present invention utilizes the comprehensive BLDG±SF formula. BLDG±SF is composed of four active ingredients [BLDG] and two dietary requirements [±SF]. The active ingredients and dietary requirements work together to help a person reliably produce equol. BLDG±SF can form the foundation of a dietary regimen composed of both supplement(s) and dietary guidelines. BLDG±SF can be viewed as a holistic treatment that has very specific scientific rationales for its parameters.

One embodiment of the present invention is a composition, for the purpose of producing equol in vivo comprising effective amounts of daidzin or daidzein, equol-producing bacteria, Camellia sinensis dry extract, and wherein the composition is provided in a pharmaceutical daily dosage form for oral administration to provide for producing the equol in vivo. In one embodiment, the pharmaceutical daily dosage form is comprised of the daidzin or daidzein in amount of about 20 to about 80 mg.

In a further embodiment, the viable equol-producing bacteria comprises bacteria from the genus Bacillus, Lactobacillus or Lactococcus.

In a still further embodiment, the pharmaceutical daily dosage is comprised of the equol-producing bacteria in an amount of about 500 million to about 20 billion CFU or the pharmaceutical daily dosage is comprised of the Camellia sinensis dry extract in an amount from about 1000 to about 3000 mg.

In an additional embodiment, the pharmaceutical daily dosage further comprises lactic acid bacteria in an amount of 5 billion to 50 billion colony forming units (“CFU”).

In an additional embodiment, a composition according to one aspect of the invention further comprises lactic acid bacteria selected from the genus Bacillus, Lactobacillus or Bifidobacterium.

In one embodiment, the pharmaceutical dosage form is selected from the group consisting of tablets, capsules and sachets containing powder for extemporaneous suspension or can comprise powder for beverages or food additives.

A still further embodiment of the invention comprises a soluble fiber or complex carbohydrate selected from the group consisting of inulin, dextrin, maltodextrin, oligosaccharide or galacto-oligosaccharides.

Another embodiment of the present invention is a method for treating climacteric and menopausal disorders affecting women in pre-, peri- and post-menopause comprising the step of administering a therapeutically effective amount of a composition comprising effective amounts of daidzin or daidzein, equol-producing bacteria, Camellia sinensis dry extract, and wherein the composition is provided in a pharmaceutical dosage form for oral administration to provide for the production of equol in vivo.

A further embodiment of the present invention is a method for treating androgenic or estrogenic hormonal disorders affecting men or women comprising the step of administering a therapeutically effective amount of a composition comprising effective amounts of daidzin or daidzein, equol-producing bacteria, Camellia sinensis dry extract, and wherein the composition is provided in a pharmaceutical dosage form for oral administration to provide for the production of equol in vivo.

An additional embodiment is a method for treating bone loss that may follow menopause or other forms of ovarian insufficiency comprising the step of administering a therapeutically effective amount of a composition comprising effective amounts of daidzin or daidzein, equol-producing bacteria, Camellia sinensis dry extract, and wherein the composition is provided in a pharmaceutical dosage form for oral administration to provide for the production of equol in vivo.

These and other objects, features and advantages of the present invention will become more apparent upon reading the following specification in conjunction with the accompanying drawing figures.

### BRIEF DESCRIPTION OF THE DRAWINGS

The accompanying drawings, which are incorporated in and constitute a part of this specification, illustrate multiple embodiments of the presently disclosed subject matter and, together with the description, serve to explain the principles of the presently disclosed subject matter; and furthermore, are not intended in any manner to limit the scope of the presently disclosed subject matter.

FIG. 1 diagrams the functional roles of the BLDG active ingredients. As shown, each of the ingredients plays multiple roles in the overall function of the formula.

FIG. 2 diagrams the entire DHT+equol lifecycle. This lifecycle includes the equol production process followed by the proliferation of equol into the body where it binds with DHT. The lifecycle ends with DHT+equol being excreted from the body through the kidneys.

FIG. 3 diagrams the amounts of daidzein and equol in bloodstream in ng/ml during the BLDG formula experimental trial period. Day 7 and Day 21 showed progressively increasing amounts of daidzein and equol in the blood.

FIG. 4 diagrams comparative amounts of BLDG formula's active ingredients for the examples, by weight.

### DETAILED DESCRIPTION OF THE INVENTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

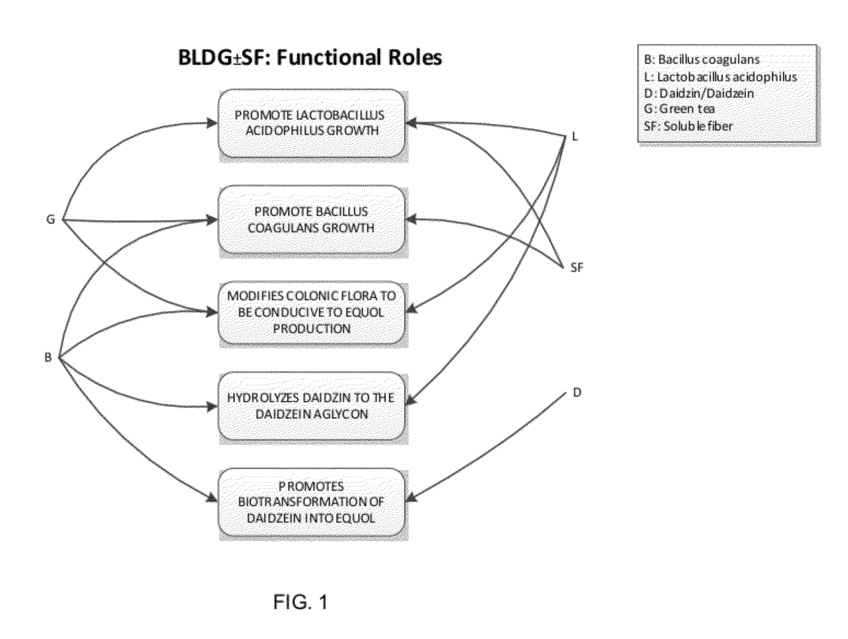

FIG. 1 diagrams the functional roles of the BLDG active ingredients. As shown, each of the ingredients plays multiple roles in the overall function of the formula.

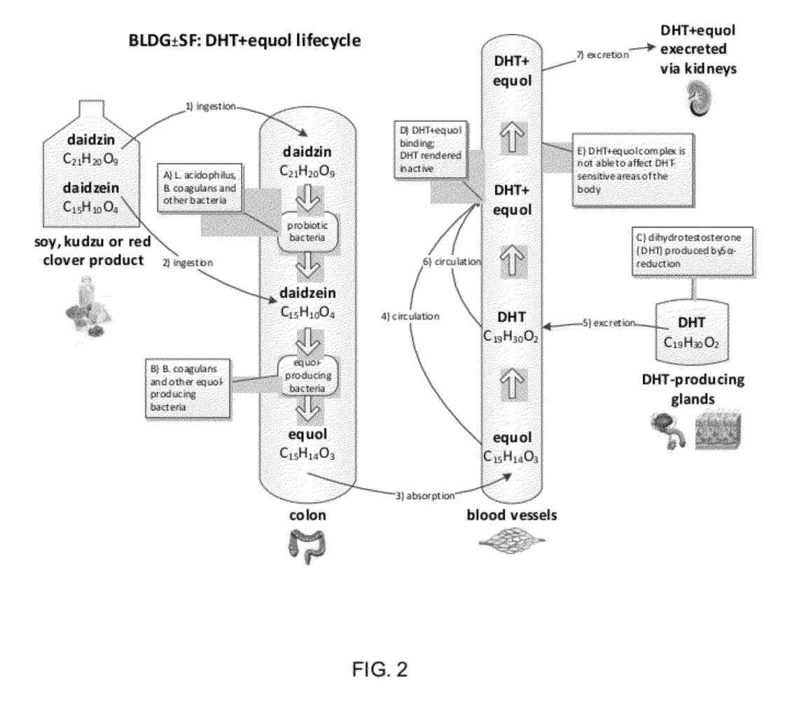

FIG. 2 is an exemplary equol production and consumption process. The following are exemplary, general steps in which a human can produce and consume equol:

2.1: Daidzin enters the digestive tract, descending through the stomach and upper intestine, finally entering the colon;

2.A: Daidzin is converted into daidzein by cleaving of the glycosidic bond. This is done by beta-glucosidases enzymes produced by L. acidophilus, B. coagulans and other probiotic bacteria;

2.2: Daidzein may enter the digestive tract directly from a food or supplement product, and thus may be ready for conversion to equol without any metabolization;

2.B: B. coagulans and any other equol-producing bacteria biotransform daidzein into equol (and O-demethylangolensin, glycetein);

2.3: Equol is absorbed through the enterocytes and enters the hepatic portal system, eventually achieving circulation;

2.4: Equol circulates throughout the body as either bound or unbound to sex hormone-binding globulin (SHBG). Unbound equol is bioavailable and therefore able to bind with DHT;

2.C: DHT is synthesized from testosterone by the 5α-reductase enzyme. Five percent of all testosterone undergoes 5α-reduction;

2.5: DHT is excreted from DHT-producing glands such as the prostate, testes and hair follicles;

2.6: DHT enters circulation and is bioavailable;

2.D: Equol binds with DHT to produce the DHT+equol complex;

2.E: The DHT+equol is biologically inactive, and has no hormonal effects. It will not bind with androgen or estrogen receptors; and

2.7: The DHT+equol complex is excreted from the body through the kidneys.

Health Benefits of Equol

Equol can provide health benefits to both men and women, but with very different results from very different mechanisms of action. For women, equol's strong affinity for the estrogen receptor can help counteract the effects of decreasing estrogen levels associated with menopause. Benefits may include: reduced frequency and severity of hot flashes, increased bone density and a reduction in muscle and joint pain. Preliminary data suggests that equol may even reduce the risk of estrogen-related cancers, including breast cancer. For men, equol exerts its primary effect by inactivating DHT. DHT is the cause of androgenic alopecia (male pattern baldness) and can aggravate acne. DHT is also a cause of benign prostatic hyperplasia (enlarged prostate) and prostate cancer; equol may help prevent these diseases. The future of equol will depend largely on the outcomes of many ongoing studies. Early indications suggest that equol has the potential to become a commonly used, highly effective dietary supplement.

Demonstrated Effects of Equol in Men

The vast majority of studies regarding equol have involved only female study participants. Research on the effects of equol in males has been limited; however, laboratory experiments involving the present invention have yielded objectively measurable quantities of equol in the blood and urine. Ongoing case results from numerous test subjects have shown that the present invention can significantly slow, halt, and even reverse the effects of androgenic alopecia in men. Given that equol has been shown to bind to, and arrest DHT, the present invention has potential to be used in the treatment of ailments of androgenic causes.

Androgenic Alopecia in Men

Androgenic alopecia is still being thoroughly researched for treatments and cures. The current scientific consensus is that DHT is the primary cause of androgenic alopecia. Genetics defines if DHT will affect the hair growth cycle of an individual. Each hair follicle produces hair in phases. The growing (anagen) phase lasts for two to six years for hair on the scalp. The cells in the root of the hair divide rapidly, adding to the hair shaft. During this phase, the hair grows about one centimeter every four weeks. After the anagen phase, a one to two week transition (catagen) phase begins. During the catagen phase, the hair follicle shrinks to about one-sixth of the normal diameter. The root is diminished and the dermal papilla breaks away. In this phase, hair growth stops, and the hair becomes a fully keratinized (dead), weak hair. The resting (telegen) phase lasts for approximately three months.

During this time, the hair is finally shed and the growth cycle restarts. It has been hypothesized that DHT causes hair follicles to enter their catagen phase prematurely, thus stunting the growth of the hair. With around 100,000 hair follicles on the head, a premature catagenic entry will result in a noticeably increased rate of hair shedding. In addition, DHT will cause each successive anagen phase to become shorter, resulting in a thinner, lighter and weaker hair. Over time, the affected hair follicles become dormant, producing no hair at all.

Hair follicles on the front and crown of the head have a higher concentration of androgen receptors to which DHT binds. This explains why the horseshoe-shaped section of the scalp is typically where male pattern baldness is observed.

The pharmaceuticals finasteride and dutasteride have been shown to reduce the levels of DHT produced in the body, therefore slowing, stopping or even reversing androgenic alopecia. Finasteride and dutasteride work as 5α-reductase inhibitors, therefore preventing DHT from being produced from testosterone. It is worth noting that although 5α-reductase inhibitors are effective, they have been known to have many side effects, such as sexual dysfunction.

Equol can work in a similar manner as the 5α-reductase inhibitors by reducing the levels of bioavailable DHT in or near the hair follicles. However, equol does not lower DHT levels in vivo, therefore avoiding the side effects of 5α-reductase inhibitors.

Producing Equol In Vivo

Current research on equol primarily focuses on the manufacturing of equol in vitro and instilling equol into supplements as an active ingredient. Various embodiments of the present invention take a different approach in that a person is given the ability to produce his or her own equol. This is the most natural, and possibly, the most effective way to deliver equol into the body. The present invention aims to emulate or enhance the natural digestive processes of a person that can normally produce equol.

Sources of Daidzin and Daidzein

The isoflavones daidzin and/or daidzein are the primary inputs required for the production of equol in vivo. Both daidzin and daidzein may be present in foods or supplements containing isoflavones, but typically daidzin is present in much greater quantities. Therefore it is more practical for the metabolization of daidzin to daidzein to occur in order for a sufficient amount of daidzein to be available in the colon for biotransformation to equol.

Soymilk as a Source of Daidzin

Previous experiments involving the present invention used unfermented soymilk as the method for daidzin/daidzein delivery. Soymilk is very rich in daidzin with 9.4 milligrams per 100 grams of soymilk. Soymilk is also readily available, easy to consume, and relatively inexpensive.

Other Sources of Daidzin and Daidzein

Other food or supplement products that provide a significant, though varying, source of daidzin and daidzein include, but are not limited to, Soy foods (tofu, tempeh, miso, etc.), Soy isoflavone extracts, Kudzu root, and Red clover

BLDG Active Ingredients

The BLDG±SF formula has four active ingredients:

[B] Bacillus Coagulans

Bacillus coagulans is a spore-forming bacterium that was initially isolated in a laboratory in 1932. It is live, does not require refrigeration, and can easily withstand passage through the stomach. B. coagulans does not colonize until it reaches the specific temperature, moisture, and enzymatic environment of the colon.

As a probiotic, B. coagulans has been used for the treatment of diarrhea (infectious, travel induced, and antibiotic induced), irritable bowel syndrome (IBS), inflammatory bowel disease (IBD) and ulcers. B. coagulans is also thought to increase generalized immune system function and digestive health. There has also been research into its role in the prevention of certain cancers.

In both conventional and alternative medicine, B. coagulans has been also used in the treatment and prevention of Candida albicans. C. albicans is an opportunistic yeast that has been known to produce thrush, vaginal yeast infections and topical yeast infections. There currently seems to be some debate in the medical community as to whether C. albicans also causes an array of digestive problems after periods of relative overgrowth.

B. coagulans is sometimes incorrectly named Lactobacillus sporogenes in dietary supplements, primarily for marketing purposes, as the Lactobacillus genus is better known for use as a probiotic.

[L] Lactic Acid Bacteria

Lactic acid bacteria (LAB), such as Lactobacillus acidophilus, are the most popular and widely used probiotics currently in use. Lactobacillus acidophilus literally means: “acid-loving, milk-bacterium” and therefore can be found in many fermented dairy products.

L. acidophilus, as our example lactic acid bacteria, is normally found in humans in the oral cavity, intestinal tract, and vagina. As a result, it has been extensively used to promote healthy digestion (prevention of both diarrhea and constipation) and the prevention of bacterial overgrowth. L. acidophilus has also been used in the treatment of diarrhea (infectious, travel induced, and antibiotic induced), irritable bowel syndrome (IBS) and inflammatory bowel disease (IBD). Patients suffering from lactose intolerance have used L. acidophilus as an adjunct to facilitate lactose digestion.

A recent study found that cattle fed with a L. acidophilus supplement had a 61% reduction in Escherichia coli 0157:H7 (the enterohemorrhagic E. coli). L. acidophilus has also been shown to increase the production of vitamin K and certain B vitamins, as well as, increase anti-microbial substances such as acidlphilin, lactocidin, acidolin and bacteriocin. L. acidophilus has also been used to protect against colon cancer.

Although L. acidophilus is the most widely recognizable member of the lactic acid bacteria genera, the group is actually much more diverse than was previously thought. The group is taxonomically categorized according to the presence of a Gram-positive, non-sporulating, rod-shaped bacilli or cocci. Members of this genera include, but are not limited to, Lactobacillus, Leuconostoc, Pediococcus, Lactococcus, and Streptococcus. The LAB are all able to withstand exposure to lower pH ranges, and thus thrive in acidic environment by being able to outcompete other, less acid-stable bacteria, in the process of natural fermentation.

This class of bacteria produces lactic as the major end product of carbohydrate fermentation and as a result, has been shown to have particular utility as probiotics. Some of the more popular LAB probiotics include, but are not limited to, L. rhamnosus, L. plantarum, L. salavarius, L. casei, and L. bulgaricus. Although Bifidobacterium is not typically included in the traditional LAB categorization (because of genetic differentiation), Bifidobacteria due indeed have a great deal of habitat overlap with traditional LAB. Like LAB, Bifidobacteria also produce lactic acid as their major metabolic end product. For this reason, Bifidobacteria may also show utility as a lactic acid producer in the BLDG formula. Widely used Bifidobacterium include B. lactis, B. bifidum and B. infantis.

[D] Daidzin/Daidzein

Daidzein is an isoflavone that also goes by the names 4,7-Dihydroxyisoflavone, daidzeol, and isoaurostatin. Daidzein is a potent antioxidant, and thus combats the damaging effects of free radicals on tissue. Isoflavones in general are thought to interfere with the proliferative stages of certain cancers by blocking uncontrolled cell growth and the formation of new blood vessels (angiogenesis). Daidzein exhibits estrogenic and anti-estrogenic effects (similar to selective estrogen receptor modulators) that may play a role in the prevention of breast cancer and maintenance of healthy hair. Daidzein has also been used in the treatment of acne.

Daidzin is the 7-O-glucoside of daidzein, and is the isoflavone form that is most prevalent in soy foods. Daidzin is a highly polar, water-soluble molecule. It is the enteric byproduct of soy metabolism. Daidzin enters the colon and is hydrolyzed by the brush border glycosidase enzymes to form the aglycon, daidzein. This chemical reaction renders the molecule less polar, less water-soluble and more bioavailable. Daidzein can then be absorbed through the enterocytes or biotransformed by a series of enzymatic reactions to produce O-demethylangolensin, glycetein and equol.

[G] Green Tea (Camellia Sinensis)

Green tea extract is a supplement that comes from the dried leaves of the Camellia sinensis shrub. Green tea has been used in the Far East for its dynamic health effects for over 5,000 years. It has been said that Prince Siddhartha Gautama (the founder of Buddhism) used green tea to help enhance his long meditations on the way to enlightenment. Green tea found its way to the western world in the 6th century via Turkish trade routes, and has been used far and wide ever since.

Green tea has been used extensively for its properties as an antioxidant. Antioxidants scavenge the body for free radicals that are generated in the normal course of daily enzymatic reactions. Antioxidants are thought to reduce cancer risks by this mechanism. Green tea extract has also been used to boost immunity. Improvement of mental performance and alertness are both benefits that have been attributed to green tea. Over the years, green tea has also been used for weight loss (both initial loss and maintenance) and also improved exercise performance and stamina. Preliminary data suggests that green tea may also lower LDL cholesterol, increase lipid metabolization and possibly effect triglyceride levels. Emerging research indicates that one of the ingredients in green tea extract may even play a role in decreasing the incidence of Alzheimer's disease.

BLDG Roles for Biotransforming Daidzin to Equol

Various active ingredients in BLDG play an important role in the overall success of various compositions according to various embodiments of the present invention. Removing any one component of BLDG has demonstrated failure in the biotransformative processes needed to produce equol in vivo. Without being held to any specific theory of operation, this is thought to be a direct result of the ingredients' effect on the colonic flora composition.

Bacillus Coagulans

B. coagulans is a lactic acid forming bacteria that aids in the conversion of daidzin into daidzein. This is achieved by cleaving the glycosidic bond by the beta-glucosidases enzyme. This cleaving of the glycosidic bond can be an important piece of the biotransformation of daidzein to equol. Daidzin, rather than daidzein, can be too polar, and thus water soluble, to get absorbed into the gastrointestinal tract. Humans do not produce beta-glucosidases naturally, so we must depend entirely on enteric probiotic bacteria, such as B. coagulans, to produce it.

Lactic Acid Bacteria Paired with Bacillus Coagulans

Similar to B. coagulans, lactic acid bacteria, such as L. acidophilus, work to fulfill the role of beta-glucosidase producing bacteria. In a recently published study, L. acidophilus showed the highest beta-glucosidase activity (in a soymilk medium) when compared with two other enteric bacteria: Bifidobacterium lactis and Lactobacillus casei.

While 32 types of cultured bacterial strains and bacterial strain combinations have been shown to biotransform isoflavones into equol in vitro, it is not completely clear which bacteria, or bacterial combinations, are responsible for this conversion in vivo. Specifically, selecting two bacterial strains with high levels of beta-glucosidase activity seemed to be a logical approach to the problem of balancing the intestinal flora in favor of equol conversion. As B. coagulans has been identified as a possible equol-producing bacterium in at least one study, B. coagulans used in conjunction with L. acidophilus seemed likely to be a strong equol-producing combination.

In July 2006, it was found (via independent laboratory confirmation) that the combination of L. acidophilus with B. coagulans was indeed able to produce significant quantities of equol in vivo. In addition, there have been significant observed effects in individuals who have consumed the BLDG±SF formula.

Green Tea (Camellia Sinensis)

Green tea polyphenols help with the biotransformation of equol through their influence on colonic flora. Green tea polyphenols were tested via growth disk method (similar to a petri dish bacterial culture, but on paper) against 45 different bacterial strains that were regarded as common intestinal microorganisms. It was demonstrated that green tea inhibits specific colonic bacteria, while promoting others. Lactic acid forming bacteria (such as L. acidophilus and B. coagulans) could stand to benefit from the effects of enhanced growth while under the influence of green tea polyphenols. Also, certain opportunistic intestinal pathogens (Clostridium difficile and Clostridium perfringens, among others) were inhibited by green tea consumption. Without opportunistic pathogens present, beneficial intestinal bacteria are not in a constant, competitive struggle to proliferate. As a result, it can be easier to cultivate a colonic flora that is much more conducive to producing large quantities of beta-glucosidase, and therefore equol.

±SF Dietary Requirements

Diet has been shown to greatly influence the body's capacity for equol production. It has been repeatedly demonstrated that the composition and metabolic activity of intestinal bacteria is strongly affected by diet. Even though the colonic flora contains over 100 trillion bacterial cells and 500 microbial species, dietary intake has been shown to significantly alter bacterial composition in as little as 24 hours.

[+SF] Additional Soluble Fiber

The lactic acid forming bacteria of the colon require a certain degree of fermenting activity in order to convert daidzein to equol. The production of lactic acid also serves the dual purpose of decreasing the surrounding pH. Lactobacilli generally thrive at a pH of 4.

Carbohydrate and fiber-rich diets promote the growth and proliferation of lactic acid producing bacteria by providing a dietary medium in which to ferment. An important factor in the rate of bacterial growth and colonization can be nutrient supply. It has been shown in one peer-reviewed study that equol-producing individuals on a conventional diet converted only 16% of daidzein into equol, whereas subjects on a carbohydrate-rich diet had a conversion rate of greater than 90%. Low-carbohydrate diets corresponded to low levels of equol production even in the presence of high daidzin/daidzein levels.

Soluble fibers have been shown to be a very effective prebiotic for the fermenting activity required of bacterium such as L. acidophilus and B. coagulans. Additionally, soluble fiber absorbs water and helps limit the movement of fecal matter to an ideal rate. Experimental results have shown the soluble fiber inulin to be a very effective prebiotic. Therefore, soluble fiber, such as inulin, can be an important part of the BLDG±SF formula in some embodiments of the present invention.

[−SF] Reduced Saturated Fats

Diets low in saturated fats also help increase intestinal fermentation, and hence equol production. Diets high in red meat (and therefore saturated fats) have been shown in rats to produce the colonic bacterial enzymes of beta-glucuronidase, azoreductase, and nitroreductase. These enzymes seem to interfere with the colonic flora and ultimately equol production. Saturated fats can have a detrimental effect on the colonic flora within 24 hours.

Individuals consuming the BLDG active ingredients while on a high fat diet typically do not achieve desired results. In addition, a full reversal in health benefits achieved from BLDG has been observed when switching from a low fat to a high fat diet.

Generally, individuals taking BLDG±SF should limit their saturated fat intake to 20 grams daily for a 2000-calorie diet.

BLDG±SF DHT+Equol Lifecycle

FIG. 2 diagrams the entire DHT+equol lifecycle. This lifecycle includes the equol production process followed by the proliferation of equol into the body where it binds with DHT. The lifecycle ends with DHT+equol being excreted from the body through the kidneys.

### EXPERIMENTAL

The goal of the first experiment, performed in June 2006, was to investigate the ability for the BLDG formula to show both objective and subjective results. Objective results included blood and urine samples testing for the presence of equol. Subjective results included the documentation of observed effects on androgenic alopecia (hair loss) and acne vulgaris.

BLDG Test Subject

The test subject was a Caucasian male, age 27, in good overall health. Subject was physically active, had adequate nutrition and was not taking any drugs or supplements that were known to cause hair loss or acne.

The subject was experiencing a high degree of shedding of the hair from areas of the scalp consistent with male pattern baldness. Miniaturization of the hair follicles on the scalp was noticeable along the hair line, part line and vertex upon close observation. The subject also had a family history of androgenic alopecia, and had been diagnosed by a general practitioner as having androgenic alopecia. The subject had previously used minoxidil 5% topically for a period of one year, which reversed some of the hair thinning. However, the minoxidil had become less effective and the shedding of hair resumed. Generally, the subject would have been considered a Class II on the Norwood-Hamilton scale, but with some increased vertex thinning more attributable to a Class III.

The subject also had persistent acne, with nodular cysts typically arising on the face, ears, shoulders, back and arms. The subject estimated the presence of about 10 moderate to severe cysts per month. The subject stated that some of these cysts were painful and scarring. The subject had not found satisfactory results from anti-acne prescription treatments since the onset of acne several years prior, with the exception of oral isotretinoin (Accutane).

Statistically speaking, the subject was unlikely to have the inherent ability to produce equol based on his Western-style diet. Before the experiment, the subject consumed soymilk over a period of four weeks without the other BLDG active ingredients as part of his diet. The trial yielded no observational results, as his hair loss continued at the same rate, and his acne persisted.

The subject then entered a two-week washout period in which he did not consume any soymilk or other products known to contain daidzin or daidzein.

BLDG Experiment

Subject consumed the BLDG formula in a regimen equivalent to Example 1 (Table 3) for a period of 21 days. The subject's diet was limited to 20 grams of saturated fat per day. The subject also consumed a healthy amount of grains, fruits and vegetables.

Blood serum and urine samples were taken on days 1, 7 and 21 and the samples were sent to an independent laboratory for analysis. At the lab, a standard HPLC/EC (High Pressure Liquid Chromatography/Electrochemical Detection) test was conducted for the determination of daidzein and equol levels in the blood serum and urine.

Equol in the blood serum was tested (untreated) as unbound to sex hormone-binding globulin (SHBG). A fraction of daidzein and equol molecules are unbound and thus able to enter a cell and activate its estrogen receptor or bind with DHT.

TABLE 1
Lab results for unbound daidzein in blood serum

| Daidzein | ng/ml I | ng/ml II | Mean | STDev |
|---|---|---|---|---|
| Day 1 (plasma) | 0.00 | 0.00 | 0.00 | 0.00 |
| Day 7 (plasma) | 790.53 | 722.41 | 756.47 | 48.17 |
| Day 21 (plasma) | 1061.31 | 1137.60 | 1099.46 | 53.95 |
| Day 21 (urine) | 44246.59 | 43701.63 | 43974.11 | 385.34 |

TABLE 2
Lab results for unbound equol in blood serum

| S-equol | ng/ml I | ng/ml II | Mean | STDev |
|---|---|---|---|---|
| Day 1 (plasma) | 0.00 | 0.00 | 0.00 | 0.00 |
| Day 7 (plasma) | 136.68 | 116.86 | 126.77 | 14.02 |
| Day 21 (plasma) | 121.52 | 141.34 | 131.43 | 14.02 |
| Day 21 (urine) | 612.75 | 662.31 | 637.53 | 35.04 |

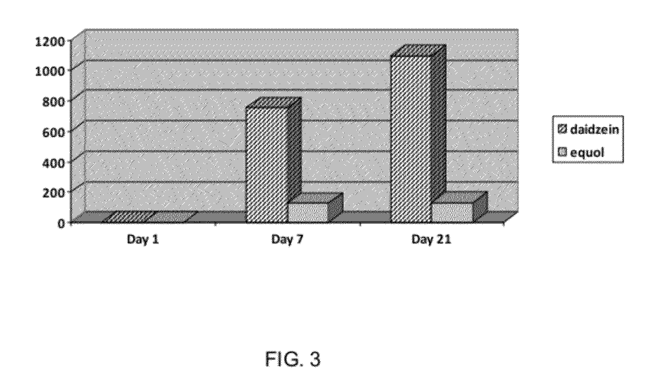

The test results are shown in Table 1 and Table 2, and a comparison of mean values for the blood serum samples are shown in FIG. 3. As anticipated, Day 1 showed no measurable amounts of daidzein and equol in the blood serum. Day 7 and Day 21 showed progressively increasing amounts of daidzein and equol in the blood. However, the difference in equol between Day 7 and Day 21 is clinically insignificant. The 45% increase in daidzein levels between Day 7 and Day 21 is likely attributable to unintentional, increased intake of soymilk before Day 21. The higher levels could also, at least in part, be the result of more efficient metabolization of daidzin to daidzein by the colonic flora.

The significant increase in daidzein levels is interesting as compared to equol, which remained much more constant. The insignificant 3% increase in equol concentration between Day 7 and Day 21 may indicate that the colonic flora hit an upper threshold in equol production rate, regardless of the amount of daidzein available. This can be thought of as a microorganism-dependent example of zero-order kinetics. An equol level of 131.43 ng/ml, as tested on Day 21, is considered a high level, as many studies deem 0.5 ng/ml to be a level where one is considered to be an equol-producer.

A recent, in vitro experiment has suggested that blood levels of 100 ng/ml may be required in order to fully mitigate DHT at normal levels of circulation in men. Therefore, the fact that BLDG can produce blood levels of unbound equol above 100 ng/ml is of great significance. Observational results showed that by Day 21, the subject had a dramatically reduced amount of hair loss as compared to Day 1, and a noticeable decrease in the amount of itchiness in his scalp. Prior itchiness was likely attributable to the accelerated shedding of the hair follicles which is caused by androgenic alopecia. In addition, the subject noticed a reduction in acne, which may have also been attributed to the presence of equol in the body. No noticeable side effects were present on or after Day 21.

BLDG Short-Term Observational Effects

Males who take the BLDG formula and have a higher rate of hair loss typically notice results very quickly. Several male test subjects have observed a significant decrease in their hair loss rate in as little as three to six weeks.

This three to six week time period may correlate with the hair growth cycle's catagen phase. If hair follicles are no longer being signaled by DHT to enter the catagen phase early, that would equate to fewer hairs being shed at a later time. Considering the catagen phase has a duration of one to two weeks, the beneficial effects from BLDG could indeed be noticeable in as little as three weeks.

Additionally, it has been noted during the trials that the type of hair being shed during the three to six week time period are generally smaller, thinner and shorter hairs. Follicles that produce healthier hairs seem to be impacted first, and miniaturized follicles also respond in time.

Males with slower hair loss or more progressed balding (Class IV or greater on the Norwood-Hamilton scale) may require a longer period of time for noticeable results. It can be concluded that BLDG will have the best short-term results for men whom are generally younger and have not yet progressed beyond a Class III.

BLDG Long-Term Observational Effects

The original test subject has continued taking the BLDG formula since June 2006 to and through March 2012. Over this period of time, the subject had observed a gradual improvement in hair thickness and texture, especially in the first few years. This suggests a slow, mild reversal of hair follicle miniaturization.

At least three other individuals have also observed a partial reversal of their hair follicle miniaturization. All of these individuals have consumed the BLDG formula for at least eight months.

The original test subject has also noticed a decrease in acne severity over the long term, with an almost complete cessation of acne outbreaks.

Utilizing the BLDG formula requires time and patience from the user in order to achieve desired results. This level of treatment-based commitment should be expected of any individual dealing with a chronic condition.

Adding Inulin (+SF) to the Diet

In November 2010, it was discovered that the addition of inulin to a regimen including the BLDG formula might be of great benefit. An individual with long-standing success with the BLDG formula was able to cut his BLDG dosage in half after starting a course of inulin supplements in the amount of 4 grams daily.

Inulin has been proven to promote growth of L. acidophilus and other probiotic bacteria while suppressing growth of E. coli and other non-beneficial bacteria. As inulin seems to greatly increase the efficacy of the BLDG formula, it is an effective prebiotic ingredient.

### EXAMPLES

The following examples each describe a possible practical application of the present invention but do not limit the invention to the examples. The BLDG±SF formula can be formulated and packaged in many different ways.

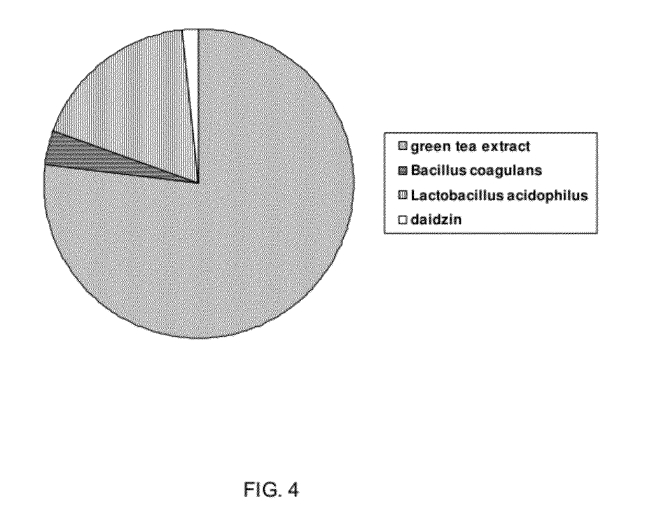

FIG. 4 illustrates the distribution of the amount of BLDG active ingredients for Example 1 and Example 2.

### Example 1

### Capsules for Oral Use

The following formulation involves the active ingredients and dietary requirements that can be used to formulate both capsules and a correlating dietary regimen under the present invention.

TABLE 3
Specification of Example 1; BLDG ± SF formulation using capsules,
soymilk, tablets

| | | |
|---|---|---|
| Component | Active Ingredient | Quantity |

In addition to a 2000 calorie diet consisting of less than 20 grams per
day of saturated fats:

| | | | |
|---|---|---|---|
| Capsule A | green tea extract | 500 | mg per capsule |
| 2 capsules twice daily | polyphenols >80% | 2000 | mg per day |
| | catechins >75% | | |
| | EGCg >45% | | |
| | caffeine <6% | | |
| | Bacillus coagulans | 50 | mg per capsule |
| | culture | 200 | mg per day |
| | 15 Billion CFU per gram | | |
| Capsule B | Lactobacillus acidophilus | 460 | mg per capsule |
| 1 capsule daily | culture | 460 | mg per day |
| between meals | 43 Billion CFU per gram | | |

Supplemental components:

| | | | |
|---|---|---|---|
| unfermented soymilk | daidzin | 21 | mg per 8 oz |
| 8 oz (227 g) twice | 9.4 mg per 100 g | 42 | mg per day |
| daily | of soymilk | | |
| soluble fiber | inulin | 4000 | mg per tablet |
| 1 tablet daily | | | |

The daily dose is contained in two capsules: Capsule A and Capsule B that contain the ingredients as shown in Table 3. The daily dose also requires the consumption of unfermented soymilk and soluble fiber, in the form of inulin. Specific amounts of the components are required for the formula to effectively produce equol as shown in Table 3. The combination of Capsule A, Capsule B, soymilk, soluble fiber and a limit on saturated fats comprises the BLDG±SF formula for this example.

The capsules are prepared without the need for any special pharmaceutical machinery or technique. The following are exemplary production steps:

1. Weighing the individual ingredients for quantity (according to Table 3)

2. Blending the ingredients with a suitable mixer

3. Filling correctly sized capsules with appropriate machinery

### Example 2

### Powder and Capsules for Oral Use

The following formulation involves the active ingredients and dietary requirements that can be used to formulate both a powdered beverage mix and capsules.

TABLE 4
Specification of Example 2; BLDG ± SF formulation using
powder/capsule
In addition to a 2000 calorie diet consisting of less than 20 grams
per day of saturated fats:

| Component | Active Ingredient | Quantity | |
|---|---|---|---|
| Powdered | green tea extract | 2000 | mg |
| Beverage Mix | polyphenols >80% | | |
| 1 scoop daily | catechins >75% | | |
| | EGCg >45% | | |
| | caffeine <6% | | |
| | Inulin | 4000 | mg |
| | Daidzin | 42 | mg |
| Capsule | Lactobacillus acidophilus | 230 | mg per capsule |
| 2 capsules daily | culture | 460 | mg per day |
| | 43 Billion CFU per gram | | |
| | Bacillus coagulans culture | 100 | mg per capsule |
| | 15 Billion CFU per gram | 200 | mg per day |

The daily dose is contained in one scoop of powder and two capsules which contain the ingredients as shown in Table 4. Specific amounts of the components are required for the formula to effectively produce equol as shown in Table 4. The combination of the powder and the capsule comprise the BLDG±SF formula for this example.

The powder and capsules are prepared without the need for any special pharmaceutical machinery or technique. The following are exemplary production steps:

1. Weighing the individual ingredients for quantity (according to Table 4)

2. Blending the ingredients with a suitable mixer

3. Filling correctly sized capsules and containers with appropriate machinery

### Example 3

### Powder for Oral Use

The following formulation involves the active ingredients and dietary requirements that can be used to formulate a powdered beverage mix under the present invention.

TABLE 5
Specification of Example 3; BLDG ± SF formulation using powder,
soymilk

| | | |
|---|---|---|
| Component | Active Ingredient | Quantity |

In addition to a 2000 calorie diet consisting of less than 20 grams per
day of saturated fats:

| | | | |
|---|---|---|---|
| Powdered | green tea extract | 1000 | mg per scoop |
| Beverage | polyphenols >80% | 2000 | mg per day |
| Mix | catechins >75% | | |
| 1 scoop | EGCg >45% | | |
| twice daily | caffeine <6% | | |
| | inulin | 2000 | mg per scoop |
| | | 4000 | mg per day |
| | Lactobacillus acidophilus culture | 115 | mg per scoop |
| | 43 Billion CFU per gram | 230 | mg per day |
| | Bacillus coagulans culture | 50 | mg per scoop |
| | 15 Billion CFU per gram | 100 | mg per day |

Supplemental components:

| | | | |
|---|---|---|---|
| unfermented | Daidzin | 21 | mg per 8 oz |
| soy milk | 9.4 mg per 100 g of soymilk | 42 | mg per day |
| 8 oz (227 g) | | | |
| twice daily | | | |

The daily dose is contained in two scoops of powder (one taken twice daily), mixed with 8 fluid ounces of unfermented soymilk for each dose, contain the ingredients shown in Table 5. Specific amounts of the components are required for the formula to effectively produce equol as shown in Table 5. The combination of the powdered beverage mix consumption, unfermented soymilk consumption, and a limit on saturated fats comprises the BLDG±SF formula for this example.

The powdered beverage mix is prepared without the need for any special pharmaceutical machinery or technique. The following are exemplary production steps:

1. Weighing the individual ingredients for quantity (according to Table 5)

2. Blending the ingredients in a suitable mixer

3. Filling containers with appropriate machinery

### Example 4

### Capsules Only for Oral Use

The following formulation involves the active ingredients and dietary requirements that can be used to formulate capsules under the present invention.

TABLE 6
Specification of Example 4 BLDG ± SF formulation using
capsules, soymilk

| | | |
|---|---|---|
| Component | Active Ingredient | Quantity |

In addition to a 2000 calorie diet consisting of less than 20 grams per
day of saturated fats:

| | | | |
|---|---|---|---|
| Capsule | green tea extract | 250 | mg per capsule |
| 8 capsules | polyphenols >80% | 2000 | mg per day |
| daily | catechins >75% | | |
| | EGCg >45% | | |
| | caffeine <6% | | |
| | Inulin | 1000 | mg per capsule |
| | | 4000 | mg per day |
| | Lactobacillus acidophilus culture | 28.75 | mg per capsule |
| | 43 Billion CFU per gram | 230 | mg per day |
| | Bacillus coagulans culture | 12.5 | mg per capsule |
| | 15 Billion CFU per gram | 100 | mg per day |

Supplemental components:

| | | | |
|---|---|---|---|
| unfermented | Daidzin | 21 | mg per 8 oz |
| soy milk | 9.4 mg per 100 g of soymilk | 42 | mg per day |
| 8 oz (227 g) | | | |
| twice daily | | | |

The daily dose is contained in four capsules taken twice daily (8 total capsules per day) which contain the ingredients shown in Table 6. Specific amounts of the components are required for the formula to effectively produce equol as shown in Table 6. The combination of the capsules, consumption of unfermented soymilk, and a limit on saturated fats comprises the BLDG±SF formula in this example.

The capsules are prepared without the need for any special pharmaceutical machinery or technique. The following are exemplary production steps:

1. Weighing the individual ingredients for quantity (according to table 6)

2. Blending the ingredients in a suitable mixer

3. Filling correctly sized capsules and containers with appropriate machinery

### Example 5

### Capsules for Oral Use

The following formulation involves the active ingredients that can be used to formulate capsules under the present invention.

TABLE 7
Specification of Example 5 BLDG ± SF formulation using capsules
In addition to a 2000 calorie diet consisting of less than 20 grams
per day of saturated fats:

| Component | Active Ingredient | Quantity | |
|---|---|---|---|
| Capsule A | green tea extract | 250 | mg per capsule |
| 8 capsules | polyphenols >80% | 2000 | mg per day |
| daily | catechins >75% | | |
| | EGCg >45% | | |
| | caffeine <6% | | |
| | Inulin | 500 | mg per capsule |
| | | 4000 | mg per day |
| | Lactobacillus acidophilus culture | 28.75 | mg per capsule |
| | 43 Billion CFU per gram | 230 | mg per day |
| | Bacillus coagulans culture | 12.5 | mg per capsule |
| | 15 Billion CFU per gram | 100 | mg per day |
| Capsule B | Daidzin | 42 | mg per day |
| 2 capsules | soy kefir powder | | |
| daily | | | |

The daily dose is contained in ten capsules. Specific amounts of the components are required for the formula to effectively produce equol as shown in Table 7. The combination of Capsule A, and Capsule B, and a limit on saturated fats comprises the BLDG±SF formula for this example.

The capsules are prepared without the need for any special pharmaceutical machinery or technique. The following are exemplary production steps:

1. Weighing the individual ingredients (according to Table 7)

2. Blending the ingredients with a suitable mixer

3. Filling corrected sized capsules with appropriate machinery

### Example Notes

While Lactobacillus acidophilus cultures were used as a representation of LAB in all example formulas, other LAB and/or Bifidobacteria may be used in place of Lactobacillus acidophilus in practice. The LAB and/or Bifidobacteria to be used must have the lactic acid fermenting capacity approximately equivalent to that of the Lactobacillus acidophilus culture quantities contained in the previously described examples. Examples of potentially suitable LAB/Bifidobacteria include, but are not limited to: L. rhamnosus, L. plantarum, L. salavarius, L. casei, B. lactis, B. bifidum, and B. infantis.

While the invention has been disclosed in its exemplary forms, it will be apparent to those skilled in the art that many modifications, additions, and deletions can be made therein without departing from the spirit and scope of the invention and its equivalents, as set forth in the following claims.

We claim:

1. A composition for producing equol in vivo, the composition comprising:
effective amounts of daidzin or daidzein;
equol-producing bacteria;
Camellia sinensis dry extract; and
wherein the composition is provided in a pharmaceutical daily dosage form for oral administration to provide for producing the equol in vivo.

2. The composition of claim 1, wherein the pharmaceutical daily dosage form is comprised of the daidzin or daidzein in amount of about 20 to about 80 mg.

3. The composition of claim 1, wherein the viable equol-producing bacteria comprises bacteria from the genus Bacillus, Lactobacillus or Lactococcus.

4. The composition of claim 1, wherein the pharmaceutical daily dosage is comprised of the equol-producing bacteria in an amount of about 500 million to about 20 billion CFU.

5. The composition of claim 1, wherein the pharmaceutical daily dosage is comprised of the Camellia sinensis dry extract in an amount from about 1000 to about 3000 mg.

6. The composition of claim 1 further comprising lactic acid bacteria selected from the genus Bacillus, Lactobacillus and Bifidobacterium.

7. The composition of claim 6, wherein the lactic acid bacteria is present in an amount of about 5 billion to about 50 billion colony forming units.

8. The composition of claim 1, wherein the pharmaceutical dosage form is selected from the group consisting of tablets, capsules and sachets containing powder for extemporaneous suspension.

9. The composition of claim 1, wherein the pharmaceutical dosage form comprises powder for beverages or food additives.

10. The composition of claim 1 further comprising a soluble fiber or complex carbohydrate selected from the group consisting of inulin, dextrin, maltodextrin, oligosaccharide and galacto-oligosaccharides.

11. A method for treating climacteric and menopausal disorders affecting women in pre-, peri- and post-menopause comprising the step of administering a therapeutically effective amount of a composition comprising:
effective amounts of daidzin or daidzein;
equol-producing bacteria;
Camellia sinensis dry extract; and
wherein the composition is provided in a pharmaceutical dosage form for oral administration to provide for the production of equol in vivo.

12. A method for treating androgenic or estrogenic hormonal disorders affecting men or women comprising the step of administering a therapeutically effective amount of a composition comprising:
effective amounts of daidzin or daidzein;
equol-producing bacteria;
Camellia sinensis dry extract; and
wherein the composition is provided in a pharmaceutical dosage form for oral administration to provide for the production of equol in vivo.

13. A method for treating bone loss that may follow menopause or other forms of ovarian insufficiency comprising the step of administering a therapeutically effective amount of a composition comprising:
effective amounts of daidzin or daidzein;
equol-producing bacteria;
Camellia sinensis dry extract; and
wherein the composition is provided in a pharmaceutical dosage form for oral administration to provide for the production of equol in vivo.
